(19) United States
(12) Patent Application Publication
(10) Pub. No.: US 2012/0160248 A1
(43) Pub. Date: Jun. 28, 2012

(54) COMPLIANT NASAL CANNULA

(75) Inventors: Robert J. McKinnon, Highlands Ranch, CO (US); James Dale Bickley, Tucson, AZ (US)

(73) Assignee: WESTMED, INC., Greenwood Village, CO (US)

(21) Appl. No.: 13/413,941

(22) Filed: Mar. 7, 2012

Related U.S. Application Data
(63) Continuation-in-part of application No. 13/232,847, filed on Sep. 14, 2011.
(60) Provisional application No. 61/382,787, filed on Sep. 14, 2010.

(51) Int. Cl. A61M 16/00

(52) U.S. Cl. 128/207.18

(57) ABSTRACT

A nasal cannula is provided that employs a left tube and a right tube wherein at least a portion thereof is made of a resilient and compliant material that elongates in response to a tensile load applied thereto. The contemplated tubing material will continue to supply a predetermined amount of oxygen to the patient if elongated, i.e., the elongated tube will not substantially constrict air flow.

This application is a Continuation-In-Part of U.S. patent application Ser. No. 13/232,847, filed Sep. 14, 2011, which claims the benefit of U.S. Provisional Patent Application Ser. No. 61/382,787, filed Sep. 14, 2010, the entire disclosure of which is incorporated by reference herein.

### FIELD OF THE INVENTION

Embodiments of the present invention are generally related to nasal cannula that deliver oxygen, a mixture of oxygen and medicine, or other gas to a patient. More specifically, one embodiment of the present invention is a nasal cannula at least partially constructed of a compliant, elastic, stretchable, or elongating material that helps decrease or prevent patient discomfort or injury.

### BACKGROUND OF THE INVENTION

Commonly, nasal cannula are comprised of left and right tubes that are interconnected on one end to a central portion that is positioned proximate a patient's nose. The other ends of the left and right tubes are interconnected to a connector that is associated with an oxygen supply by way of another tube. The left tube, right tube, and central portion is often referred to as a “harness.” The central portion includes two nares that are designed for insertion into a patient's nostrils. It is important to maintain the location of the central portion and the nares relative to the patient's nostrils to ensure the desired amount of oxygen is delivered to the patient. The most common way to achieve and maintain proper placement of the central portion is to position the left and right tubes around the patient's ears which creates an upward tension on the tube. An adjustable bolo tie is used to selectively adjust the size of the loop of tubing formed by the left and right tubes under the patient's chin. This ensures proper tension of the tubing between the patient's ears and thus maintains placement of the central portion relative to the patient's nostrils.

When nasal cannula are used for short periods of time, placement of the left and right tubes over and behind the patient's ears is ideal. However, patients afflicted with long-term chronic respiratory conditions, such as emphysema, must regularly or continuously receive oxygen by way of nasal cannula and often experience ear, cheek, or neck irritation and discomfort. Similarly, patients with less resilient skin, such as the elderly, can experience discomfort and injury to the skin from tubing rubbing on their skin at a much higher frequency or incident rate. That is, portions of the left and right tube will continuously contact and rub the patient's ear and neck which often causes discomfort and injury. In some situations, cannula can cause contact ulcers and wounds, which may be severe that lead to infection and possibly death. Moreover, once a sore or ulcer is created, the more delicate skin of elderly patients is slower to recover and less likely to heal if the tubing remains present. In addition, normal head tilting or rotation will increase the tension applied to either the left or right tube depending upon the direction of rotation which increases pressure on a patient's ear. Further, any tension increase will increase pressure on the ear if a bolo tie is used. The increase in tension may also increase in the pressure applied to the patient's cheek, or neck. Head turning will also allow one tube to slacken, which may allow the nares to fall from the patient's nostrils. These problems have plagued patients for years, yet no successful solution has been developed.

More specifically, in order to address ear and neck chafing, ointments and pads have been employed to prevent direct engagement of the tubes with the patient. For example, a product of the assignee of the instant application, EarMates™, are soft and compliant foam members that are positioned around the portion of the left and right tubes that are located over and behind the patient's ears. Other products support the tubing with clips that interconnect to the patient's glasses or hat. Although such devices eliminate tube-to-ear tube contact, they are cumbersome and are useless if the patient does not wear glasses or a hat. In addition, such devices cannot easily be used when the patient is sleeping.

U.S. Pat. No. 4,699,139 to Marshall and U.S. Pat. No. 5,025,805 to Nutter, disclose other ways of addressing user discomfort. Marshall provides small pads that are placed about portions of the tubes located behind the patient's ear. Nutter employs an elongated pad that covers portions of the tube from a point behind the patient's ear and along the patient's cheeks. Although these devices isolate static or dynamic loads generated by a change in the orientation of the patient's head, they are visually unappealing and often deflect the patient's ear outwardly. More specifically, the very nature of these devices is that they have an outer diameter that is greater than that of the tube which allows the devices to spread pressure loads over a larger area. Thus these devices deflect the patient's ear outwardly, which may cause discomfort. Furthermore, sleeping with pads or similar devices positioned behind the ears is generally difficult, if not impossible, for many patients as impingements and pressure spots are created when the patient sleeps on his or her side. Further, although soft, these devices can cause discomfort when the patient moves his or her head as the pads will frictionally engage and irritate the outside of a user's ear, their neck, and/or their face. Moreover, once a sore or ulcer is created, the pad causes further irritation and does not permit healing. In addition, the pads are difficult to install properly and often become soiled and fouled by patient perspiration.

Other cannula have attempted to address the issue of static and dynamic pressure by providing tubes that slide relative to fixed ear pieces. U.S. Patent Application Publication No. 2004/0035431 to Wright, for example, discloses such an ear piece. Wright, however, has the same drawbacks as the devices described above wherein the earpieces are much larger than the tubes. Thus the ear pieces will deflect the user's ear outwardly causing discomfort and similarly create discomfort when a patient is positioned on his or her side thereby making sleeping almost impossible. Further, when the tubes are installed within the earpieces, a predetermined amount of slack must be provided to allow the patient's head to move comfortably, which is difficult to implement as the slack also makes it difficult to maintain the position of the central portion relative to the patient's nose.

Some have addressed patient discomfort by making the cannula harness of a soft or compliant material, such as silicon. One drawback of using silicon is that it can be very expensive. More specifically, silicon, which is generally liquid in its raw form, requires special manufacturing and processing equipment which adversely affects the manufacturing cost of silicon cannula. Further, primers and special adhesives are required to bond silicon to the materials that the central portion and connector of the harness are made, which adds manufacturing complexity and cost. Even if all of these drawbacks, and other drawbacks understood to those in the art concerning the use of silicon, are addressed, the silicon currently used does not perform as well as the embodiments of the present invention described below.

A long felt need exists to provide a nasal cannula that is comfortable to wear and yet allows an individual to wear the cannula for extended periods of time and turn their head without creating pressure points that will eventually cause or exacerbate discomfort or injury. The following disclosure describes an improved nasal cannula that employs tubes at least partially constructed of a resilient material which elongates when tension is applied without restricting air flow through the tubes.

### SUMMARY OF THE INVENTION

Nasal cannula are commonly comprised of a central portion having a pair of nares that fit within the nostrils. A left tube, which is placed over the left ear of a patient and a right tube, which is placed over the right ear of the patient, extend from the central portion. The tubes are then directed beneath the patient's jaw line and around the front of the patient's neck to a juncture interconnected to a gas delivery line by way of a connector. In order to ensure that the left tube and right tube are firmly secured to the patient's head, a bolo tie is often employed that allows the tension of the left tube and right tube to be selectively altered by increasing or decreasing the amount of tubing between the bolo tie and the central portion. One drawback of traditional nasal cannula is that tensioning the bolo tie necessarily increases the pressure and frictional load applied to the patient's face and ears such that when the patient tilts or rotates their head, their skin becomes irritated. Relieving some of the tube tension by loosing the bolo, however, may allow the central portion to fall from the nostrils and does not address the static pressure applied to a patient's skin by the tubing.

It is thus one aspect of the present invention to provide a nasal cannula made at least partially of a flexible material that readily elongates without restricting air flow. In one embodiment, the left and right tubes are made at least partially of a material that is capable of stretching approximately 1 inch per foot of length when less than about 0.5 pounds of force is applied. The length increase reduces tube tension and associated pressure, thereby reducing or eliminating patient irritation. One embodiment of the present invention is made of an elastomer, such as flexible polyvinyl chloride (PVC), polyurethane, or similar material which elongates when tensioned without reducing the internal diameter to such a degree that would constrict the gas flow. For example, the left and right tubes may be at least partially constructed of a material having a shore hardness of about 50, however, materials of various hardness may be used without departing from the scope of the invention. One of skill in the art will appreciate that the left and right tubes may only include a segment of such resilient material rather than constructing the entire tube of such material. It is contemplated that such segment would at least match or align with the length of tubing that would interface with the patient's ear. The elongating portion of the tube may be fused or otherwise interconnected to stiffer portions of the cannula that do not contact the patient's face and neck.

Further, one embodiment of the present invention employs left and right tubes made of flexible PVC formed of molded or extruded pellets. The left and right tubes are bonded to the central portion and the connector, which may be made of a material different from that of the left and right tube, without primers. For example, the left tube, right tube, central portion, and connector of one embodiment are made of a form of PVC (i.e., not silicone) and bonded with butanone (also known as methyl ethyl ketone or MEK) or cyclohexane.

Still other embodiments of the present invention employ a bellows positioned adjacent to the patient's ear that selectively elongates in response to head movement.

It is another aspect of the present invention to provide a nasal cannula assembly designed for contact with the nasalabidial area of a patient's nose and comprising, consisting essentially of, or comprised of: a hollow tubular member having an oxygen supply opening at each end and having a pair of spaced tubular extensions projecting therefrom that terminate in gas-directing orifices; a first tube interconnected at a first end to one end of the hollow tubular member, the first tube is made of a material that stretches about 1 inch per foot of length when less than about 0.5 lbs of tension is applied thereto; and a second tube interconnected on a first end to an end of the hollow tubular member opposite from where the first tube is connected, the second tube is made of a material that stretches about 1 inch per foot of length when less than about 0.5 lbs of force is applied.

It is still yet another aspect of embodiments of the present invention to provide a nasal cannula comprising, consisting essentially of, or comprised of: a first tube adapted for positioning behind a patient's left ear; a second tube adapted for positioning behind a patient's right ear; a central portion interconnected to the first tube and the second tube, the central portion having at least one branch for insertion within a patient's nose; and wherein all or at least a portion of the first tube and the second tube employs a material that elongates in response to movement of the patient's head.

The Summary of the Invention is neither intended nor should it be construed as being representative of the full extent and scope of the present invention. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description of the Invention and no limitation as to the scope of the present invention is intended by either the inclusion or non-inclusion of elements, components, etc. in this Summary of the Invention. Additional aspects of the present invention will become more readily apparent from the Detail Description, particularly when taken together with the drawings.

### BRIEF DESCRIPTION OF THE DRAWINGS

The accompanying drawings, which are incorporated in and constitute a part of the specification, illustrate embodiments of the invention and together with the general description of the invention given above and the detailed description of the drawings given below, serve to explain the principles of these inventions.

FIG. 1 is a perspective view of an individual wearing a nasal cannula of one embodiment of the present invention;

FIG. 2 is a left elevation view of the embodiment shown in FIG. 1;

FIG. 3 is a schematic of FIG. 2 showing a cross section of the central portion and cross sections of the tube in a relaxed and stretched state;

FIG. 4 is a perspective view of another embodiment of the present invention that employs flexible bellows;

FIG. 5 is a schematic of FIG. 4 showing cross sections of the bellows in a relaxed and stretched state;

FIG. 6 is a free body diagram showing the forces acting on a stretched tube.

It should be understood that the drawings are not necessarily to scale. In certain instances, details that are not necessary for an understanding of the invention, or that render other details difficult to perceive, may have been omitted. It should be understood, of course, that the invention is not necessarily limited to the particular embodiments illustrated herein.

### DETAILED DESCRIPTION

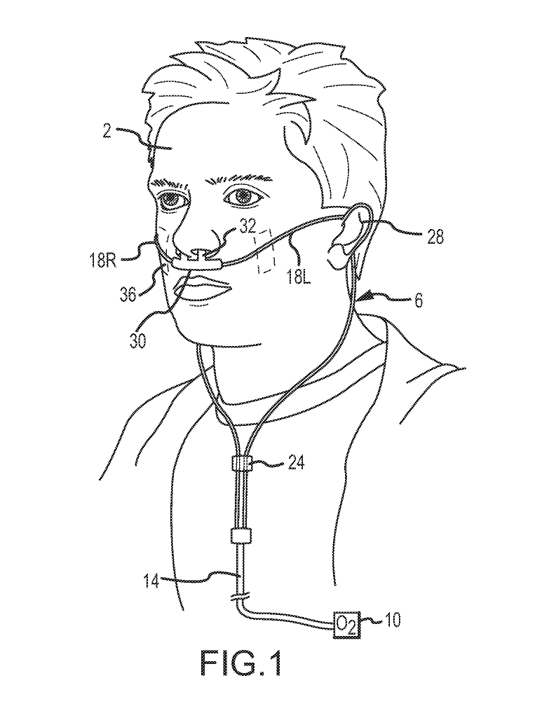

FIG. 1 shows a patient 2 wearing a nasal cannula 6. The nasal cannula 6 is interconnected to an oxygen supply source 10 and includes an oxygen tube 14 that splits into a right tube 18R and a left tube 18L. The right tube 18R and the left tube 18L are tightened to the patient's head by sliding a bolo type tie 24 or other device towards the patient's chin. The right tube 18R and the left tube 18L are placed around the patient's ears 28 such that a central portion 30 with oxygen delivering nares 32 are located securely within the patient's nostrils. In order to further secure the nasal cannula, tape 36 may be employed that is affixed to the patient's cheek, for example.

Although FIG. 1 shows a commonly used oxygen delivery cannula, the aspects of the present invention described herein may be incorporated into a cannula that supplies oxygen and monitors CO2 and oxygen demand. More specifically, in some instances the cannula nares 32 are separated by a wall positioned within the central portion 30, wherein one of the tubes is interconnected to an oxygen supply and one of the tubes is interconnected to a CO2 or oxygen demand monitor. The wall prevents intermingling of the oxygen taken in by the patient and CO2 expelled from the patient. The tube associated with the oxygen supply may be of a larger diameter than the tube associated with the CO2 or oxygen demand monitor. In some instances the smaller tube may be positioned within a larger diameter tube wherein at least a portion of both tubes are flexible. In operation, the CO2 monitor draws in a portion of the exhaled air to measure the amount of CO2 present. An oxygen demand monitor is used to conserve oxygen by measuring the pressure within the tube to which it is interconnected and upon a drop in pressure, which indicates inhalation, delivers oxygen to the patient.

As patients sometimes exhale out their mouth instead of their nose, a CO2 monitor interconnected to the central portion 30 as described above may not detect exhaled CO2. To address this issue, on oral branch may be employed that connects the central portion 30 to the CO2 monitor. The oral branch has one end in fluid communication with the nare interconnected to the CO2 monitor and another end that is positioned near the mouth. Thus, when the patient exhales, regardless of whether it is from their nose or mouth, exhaled air will travel to the CO2 monitor.

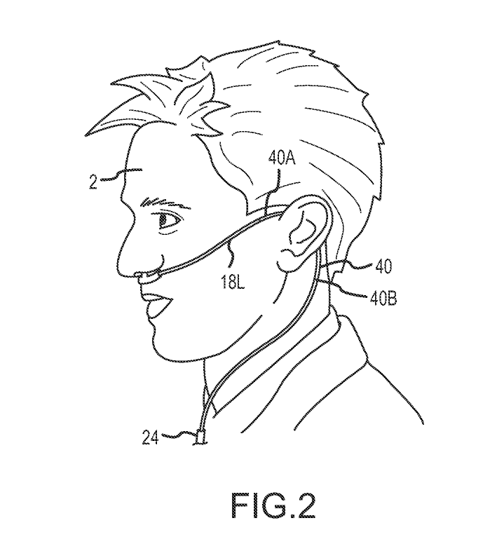

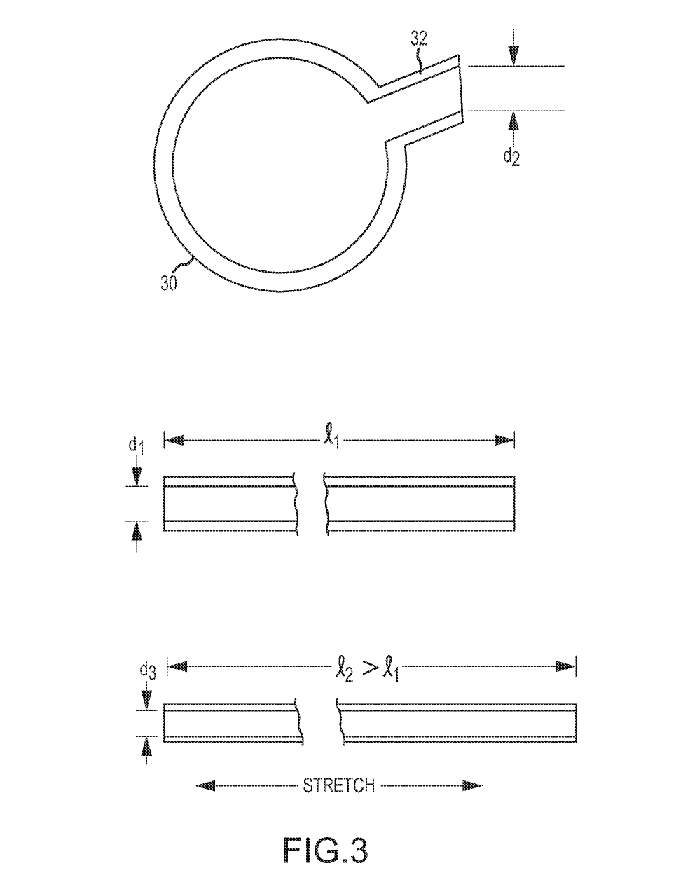

Referring now to FIGS. 2 and 3, an ear portion 40 associated with the left tube 18L and the right tube 18R are made of a compliant and resilient material that is capable of elongation without adversely affecting the flow of gas therethrough. Using the left side of the patient's head for illustrative purposes, the compliant portion begins at point 40a and ends at point 40b. One of skill in the art will appreciate that the compliant portion can extend from the nose piece to the bolo tie 24, i.e. the entire length of the left tube 18L and the right tube 18R, or may extend for a portion of the length of the left and right tubes. In a cannula of this construction, the non-compliant portion of the oxygen tube may be made of a stiffer, kink-resistant material, such as PVC with a shore hardness of 65 to 70, i.e., a material that is different from that of the compliant portions. Further, in one embodiment the left tube 18L and the right tube 18R are not comprised of silicone. The ear portion employs tubing with an inside diameter d₁ larger than the inside diameter d₂ of the nose piece nares 32. The elongation of the left tube 18L and/or the right tube in response to head tilting and turning are such that elongations of up to 10% of the tubing length will not decrease the tube's inside diameter d₃ to a dimension smaller than the openings of the nares d₂.

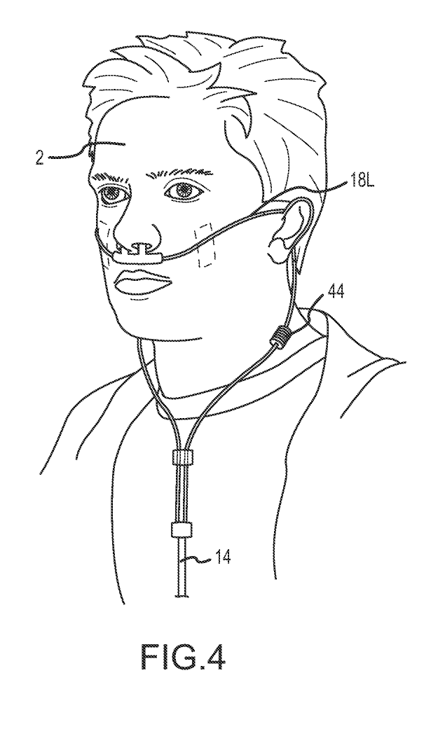

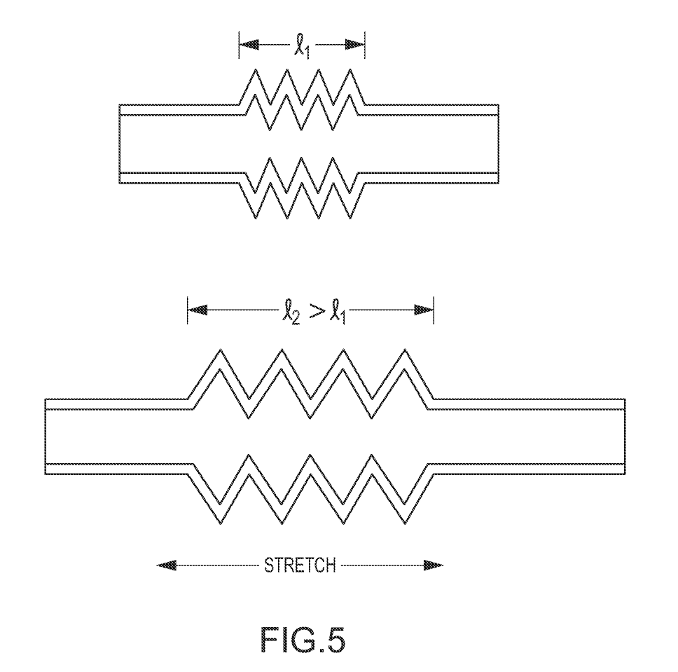

As shown in FIGS. 4 and 5, other embodiments of the present invention employ left and right tubes with a bellows section 44 that allows for the left tube and the right tube to expand to accommodate head movement. The bellows 44 may be positioned between the nose and ear, or between the ear and bolo tie. During movement, the bellows expands and contacts in length appropriate.

Testing was performed to assess the functionality of embodiments of the present invention. More specifically, a cord was used to determine the change in distance from an individual's nose to the top of their ear as they moved their head from a forward position to a fully turned, rotated position. Initially, the cord was held against an individual's nose and the top of their ear. The point of contact between the cord and the patient's ear was then noted. The individual was directed to turn his or her head as far as possible in the direction opposite from the ear in contact with the cord. As the individual's head was turned the cord was allowed to slide relative to the ear but was fixed relative to the nose. This simulated the amount of tube stretch needed to accommodate a patient turning his or her head, which allowed assessment of the amount of tube stretch needed to reduce or eliminate the application of force by left and right tubing onto a patient's head due to head turning. The point of contact of the cord and the individual's ear was noted after head rotation. The distance between the first ear contact point and the second ear contact point was then measured. Similar measurements were obtained from four other individuals and an average percentage distance change was calculated.

Next, a three inch section of standard tubing was interconnected to a tensile force tester wherein one inch of tubing was held within the jaws of the force tester. The tubing was then stretched at a rate of ten inches per minute and the tension noted when the tubing was elongated to a distance equal to the average percentage distance change described above. Twenty tubing samples were tested in this manner.

This test yielded an average tube length increase of 10% associated with a full head rotation. Further, it was found that nasal cannula made by Vapotherm® experiences a tensile force of about 3.0 psi when the tubing is elongated by 10% of its length at a rate of 10 inches per minute. As one of skill in the art will appreciate, the amount of tensile force applied to the tubing is directly proportional to the force felt by the patient and the associated injury and/or discomfort. The tensile force was reduced to about 0.1 psi when a cannula with ear pieces made of flexible PVC, which is capable of elongation up to 450% of its original length without being damaged or permanently deformed, was tested. “Flexible PVC” is a general term that refers to a material comprised of rigid PVC combined with plastic resins, plasticizers, or any agent that renders the material soft, flexible, and able to elongate and return to its original shape. For example, the clear medical grade flexible PVC APEX®3200-50NT manufactured by Teknor Apex may be used, which has a Shore A hardness of 50 and elongation of 480%. Because human facial tissue resilience and facial structure and shape vary from patient-to-patient, the results of this test cannot be used to determine the actual amount of pressure applied to a patient's ears, nose, and face. However, it is clear from the test that the embodiments of the present invention will exert far less force on the patient's ears than the Vapotherm® tubing or tubing having similar characteristics. Further, it is believed that any elastomer that would exert less than 3.0 psi, preferably less than about 1.5 psi, and most preferably less than 0.5 psi, under the test conditions described above would help reduce or eliminate patient discomfort and injury. In addition, although flexible PVC capable of elongation up to 450% of its original length has been described, one of skill in the art will appreciate that a flexible PVC capable of elongation of approximately 250% or more may be used without departing from the scope of the present invention. Further, flexible PVC capable of elongation more than 450% of its original length may be used in some instances.

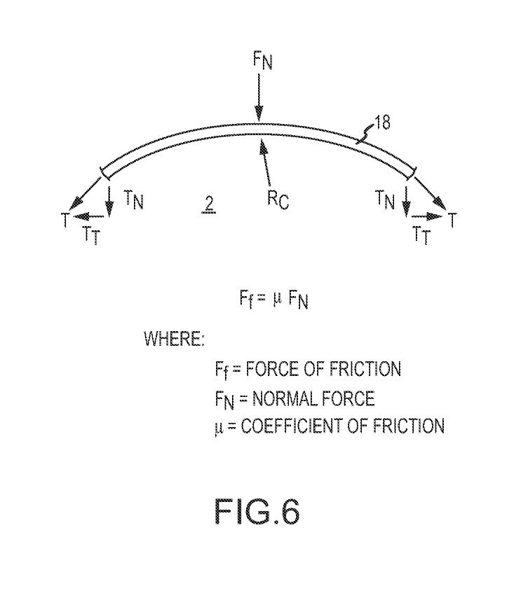

As briefly mentioned above, the amount of tensile force on the tube 18 is directly proportional to the amount of frictional force felt by the patient 2. That is, a cannula made in accordance with embodiments of the present invention will exert a decreased level of normal force onto a patient's face and/or ear. The normal force is proportional to the amount of pressure and friction generated by the moving tube which can cause patient discomfort. FIG. 6 is a free body diagram showing the forces generated by a tensioned tube 18 on a curved surface, such as a face or an ear. In the context of embodiments of the present invention, the tube 18 is engaged on a patient's 2 face and/or ear, wherein (R_{c}) represents a radius of curvature that generally corresponds to the shape of the patient's face. When the tube 18 is tensioned (T), a force normal (F_{N}) to the patient's face, which is proportional to the normal component of the tension (T_{N}), will be generated. As will appreciated by those of skill in the art, the normal component (T_{N}) and tangential component (T_{T}) of the tension, and thus the normal force (F_{N}), will be dependent on the shape of the patient's physical features (R_{c}). Thus embodiments of the present invention reduce the amount of frictional force and pressure applied to the patient's head by drastically decreasing the amount of tension (T) generated when the tube 18 is pulled.

Although flexible PVC has been described, one of skill in the art will appreciate that other flexible or resilient materials having characteristics equivalent to flexible PVC as explained herein, may be used provided appropriate medical standards are also satisfied. More specifically, any material with an elongation that will render such material soft and stretchable yet resilient such that it will return to its original condition after tension in the ranges applied in the context of the present invention is removed is within the scope of the invention. That is, any extrudable material capable of elongating more than 300% of its original length without permanently deforming or rupturing while maintaining necessary gas delivery flow rates may be used. For example, tubes made at least partially of silicon or polyurethane would suffice, but these materials are often more expensive than flexible PVC and thus not as desirable. Further, the tubes may be made of a combination of suitable materials or a combination of currently-used materials with segments comprised of suitable materials.

While various embodiments of the present invention have been described in detail, it is apparent that modifications and alterations of those embodiments will occur to those skilled in the art. However, it is to be expressly understood that such modifications and alterations are within the scope and spirit of the present invention, as set forth in the following claims. Further, the invention(s) described herein is capable of other embodiments and of being practiced or of being carried out in various ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

What is claimed is:

1. A nasal cannula assembly designed for contact with the nasalabidial area of a patient's nose and comprising:
a hollow tubular member having an oxygen supply opening at each end and having a pair of spaced tubular extensions projecting therefrom that terminate in gas-directing orifices;
a first tube interconnected at a first end to one end of said hollow tubular member, said first tube is made of a material that stretches about 1 inch per foot of length when less than about 0.5 lbs of tension is applied thereto; and
a second tube interconnected on a first end to an end of said hollow tubular member opposite from where the first tube is connected, said second tube is made of a material that stretches about 1 inch per foot of length when less than about 0.5 lbs of force is applied.

2. The nasal cannula assembly of claim 1, wherein said first tube and said second tube are not made of silicone.

3. The nasal cannula assembly of claim 1, wherein said first tube and said second tube are made of at least one of flexible PVC and polyurethane.

4. The nasal cannula assembly of claim 1, wherein said an inside diameter of said first tube and an inner diameter of said second tube are larger than the inside diameter of said spaced tubular extensions.

5. The nasal cannula assembly of claim 1, wherein said first tube and said second tube does not decrease below a predetermined diameter when at least one of said first tube and said second tube are elongated by up to 10%.

6. The nasal cannula assembly of claim 1, wherein said material of said first tube and said second tube is confined to a portion thereof that is adapted to be positioned around the patient's ear.

7. The nasal cannula assembly of claim 1, wherein said first tube is adapted to be positioned behind a patient's left ear and said second tube is adapted to be positioned behind a patient's right ear wherein said first tube will apply less than about 1.5 psi to the patient's face or neck when the patient's head is turned to the right and wherein said second tube will apply less than about 1.5 psi to the patient's face or neck when the patient's head is turned to the left.

8. The nasal cannula assembly of claim 1, wherein said first tube is adapted to be positioned behind a patient's left ear and said second tube is adapted to be positioned behind a patient's right ear wherein said first tube will apply less than about 0.1 psi to the patient's face or neck when the patient's head is turned to the right and wherein said second tube will apply less than about 0.1 psi to the patient's face or neck when the patient's head is turned to the left.

9. The nasal cannula assembly of claim 1, wherein a 10% increase in the length of said first tube and a 10% increase in the length of said second tube will generate a tensile force of less than 1.5 psi in said first tube and said second tube.

10. The nasal cannula assembly of claim 1, wherein said first tube and said second tube are interconnected to said hollow tubular member without primers.

11. The nasal cannula assembly of claim 1, wherein said first tube and said second tube are made of a material that possesses an elongation of about 250% or more.

12. A nasal cannula comprising:
a first tube adapted for positioning behind a patient's left ear;
a second tube adapted for positioning behind a patient's right ear;
a central portion interconnected to said first tube and said second tube, said central portion having at least one branch for insertion within a patient's nose; and
wherein said first tube and said second tube employs a material that elongates in response to movement of the patient's head, said first tube and said second tube experiencing less than about 1.5 psi of tensile force when elongated 10% of its original length.

13. The nasal cannula of claim 12 wherein said first tube is made of a material that stretches about 1 inch per foot of length when less than about 0.5 lbs of tension is applied thereto; and
said second tube is made of a material that stretches about 1 inch per foot of length when less than about 0.5 lbs of tension is applied thereto.

14. The nasal cannula assembly of claim 12, wherein said first tube and said second tube are not made of silicone.

15. The nasal cannula of claim 12, wherein said first tube and said second tube are made of flexible polyvinyl chloride or polyurethane.

16. The nasal cannula of claim 12, wherein the diameter of said first tube and the diameter of said second tube does not decrease below a predetermined diameter when at least one of said first tube and said second tube are elongated by 10% of its original length.

17. The nasal cannula of claim 12, wherein said first tube will apply less than about 1.5 psi to the patient's face or neck when the patient's head is turned to the right and wherein said second tube will apply less than about 1.5 psi to the patient's face or neck when the patient's head is turned to the left.

18. The nasal cannula assembly of claim 12, wherein said first tube is also interconnected to an oxygen source and said second tube is also interconnected to a carbon dioxide monitor or an oxygen delivery monitor.

19. The nasal cannula assembly of claim 12, wherein said material has an elongation of about 250% or more.

20. The nasal cannula assembly of claim 12, wherein a 10% increase in the length of said first tube and a 10% increase in the length of said second tube will generate a tensile force of less than 1.5 psi in said first tube and said second tube.
